(19) United States
(12) Patent Application Publication
(10) Pub. No.: US 2009/0209548 A1
(43) Pub. Date: Aug. 20, 2009

(54) FORMULATION FOR STIMULATING HAIR GROWTH

(75) Inventors: Susan Marie Ciotti, Ann Arbor, MI (US); Darshan K. Parikh, Bridgewater, NJ (US)

Correspondence Address: PFIZER INC.;PATENT DEPARTMENT, Bld 114 M/S 114, EASTERN POINT ROAD, GROTON, CT, 06340, US

(73) Assignee: Pfizer Inc

(21) Appl. No.: 12/367,939

(22) Filed: Feb. 9, 2009

Related U.S. Application Data
(63) Continuation of application No. 11/572,186, filed on Jan. 16, 2007, now abandoned.
(60) Provisional application No. 60/589,126, filed on Jul. 19, 2004.

(51) Int. Cl. A61K 31/501; A61P 43/00

(52) U.S. Cl. 514/252.01

(57) ABSTRACT

The present invention is directed to a topical formulation comprising the Compound 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone in a formulation comprising at least one pharmaceutically acceptable carrier. Particularly, the formulation may be used to promote hair growth, including alleviating alopecia.

### FIELD OF THE INVENTION

The invention is directed to topical formulations for delivery of the compound, 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone. These formulations are useful for promoting hair growth.

### BACKGROUND OF THE INVENTION

U.S. Pat. No. 5,912,244 discloses the compound 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone (hereinafter “the Compound”), methods for its preparation and its use as a potassium channel opener.

Co-pending, commonly assigned, U.S. Patent Application Ser. No. 60/544,116, filed Feb. 12, 2004, discloses the topical application of the Compound to promote hair growth in humans. Animal studies have demonstrated that the Compound alters the hair growth cycle by inducing anagen. Anagen is the growth phase, during which the follicle (i.e. the hair root) penetrates deep into the dermis with the cells of the follicle dividing rapidly and differentiating. During the anagen phase, the hair cells synthesize keratin, the predominant proteinaceous component of hair. In non-balding humans, anagen lasts from one to five years. Catagen is the transitional phase of the cycle and is marked by the cessation of mitosis. Catagen generally lasts from about two to three weeks. Telogen is the resting phase of the cycle in which the hair is retained within the scalp for up to 12 weeks, until it is displaced by new follicular growth from the scalp below. In healthy young humans, most of the hair follicles will be in the anagen phase. In such individuals, the anagen to telogen ratio can be as high as 9 to 1. In individuals with alopecia, this ratio is reduced to as low as 2:1.

The skin consists of three main layers, the epidermis, the dermis and the subcutaneous fat layer. The epidermis comprises the stratum corneum and the viable epidermis. The stratum corneum, the most external layer of the epidermis, is composed primarily of keratinized dead cells. It is the major barrier to the penetration of externally applied materials through the skin. The dermis consists of a matrix of connective tissue, which is penetrated by blood vessels, nerves and skin appendages. The hair follicle, which is where anagen is initiated, is located deep in the dermis. FIG. 1 illustrate these three layers and the hair follicle's relative position in the skin.

To initiate anagen, the Compound must penetrate the stratum corneum, the viable epidermis and much, if not all of, the dermis in order to reach the hair follicle. Formulations that would enhance the Compounds penetration thru the stratum corneum, into the dermis and ultimately to the follicle would be expected to enhance the activity of the Compound.

### SUMMARY OF THE INVENTION

In accordance with the present invention, topical formulations providing for the enhanced delivery of 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone are provided. These formulations increase the absorption of the Compound by the skin. These formulations provide for increased concentrations of Compound in the dermis. It would be expected that these enhanced concentrations should provide a more effective treatment for alopecia, by enhancing the rate of hair growth.

The invention is directed to a topical formulation comprising (a) the Compound, (b) a dermatologically acceptable carrier, and (c) said formulation exhibits a flux of said Compound across human cadaver skin into a receptor compartment of a Franz Diffusion cell that is at least three times higher than the flux exhibited by a reference formulation (i.e. 70% ethanol/30% propylene glycol w/w).

The topical formulation may be in the form of aqueous, alcoholic or aqueous-alcoholic solutions, or in the form of creams, gels, emulsions or mousses, or alternatively in the form of aerosol compositions also comprising a propellant under pressure. The composition according to the invention can also be a hair care composition, and in particular a shampoo, a hair-setting lotion, a treating lotion, a styling cream or gel, a dye composition, a lotion or gel for preventing hair loss, etc.

The quantity of Compound present in the topical formulation can vary, provided it is sufficient to promote hair growth (i.e. an effective amount). Typically, the Compound will be present in the quantity of from about 0.001 to about 10% (w/w). It typically will be administered from 1 to 4 times daily, or less frequently for instance once a week.

The formulation will typically be used to alleviate alopecia, especially androgenic alopecia. In a further embodiment, the invention is directed to an article of manufacture containing the topical formulation, packaged for retail distribution, in association with instructions advising the consumer how to use the product to promote hair growth. (i.e. a kit)

### BRIEF DESCRIPTION OF DRAWINGS

FIG. 1 depicts the structure of skin.

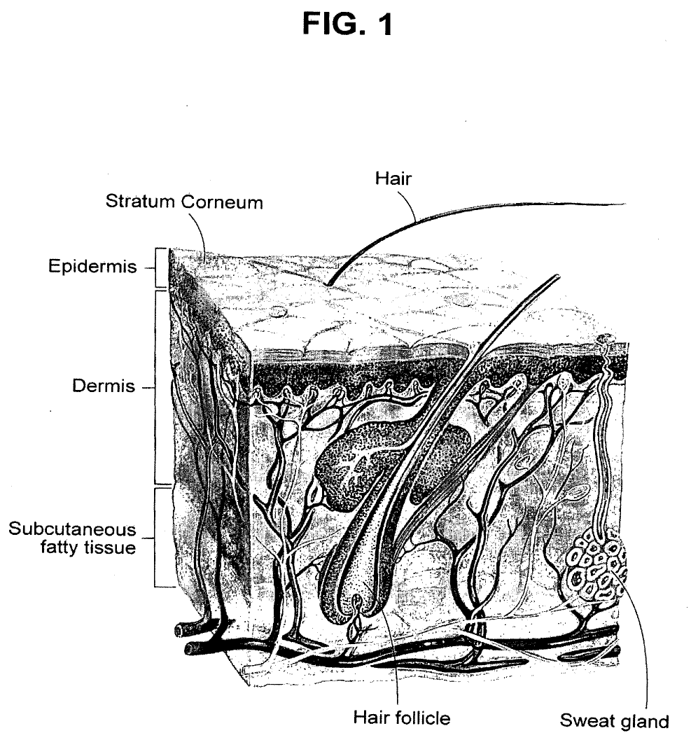

FIG. 2 depicts a Franz cell diffusion device.

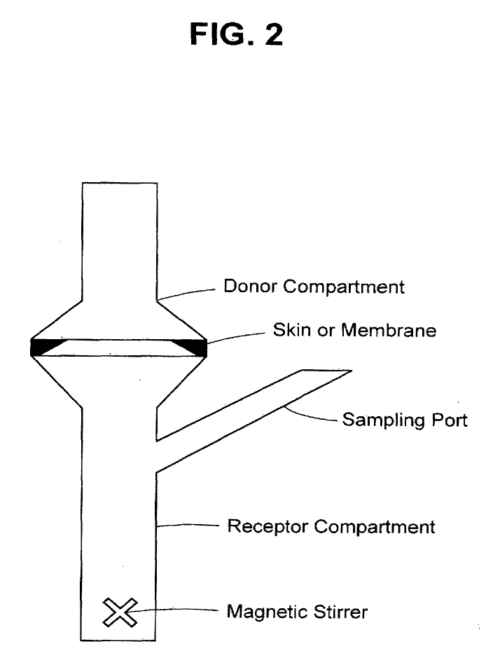

FIG. 3 depicts a Flux calculation.

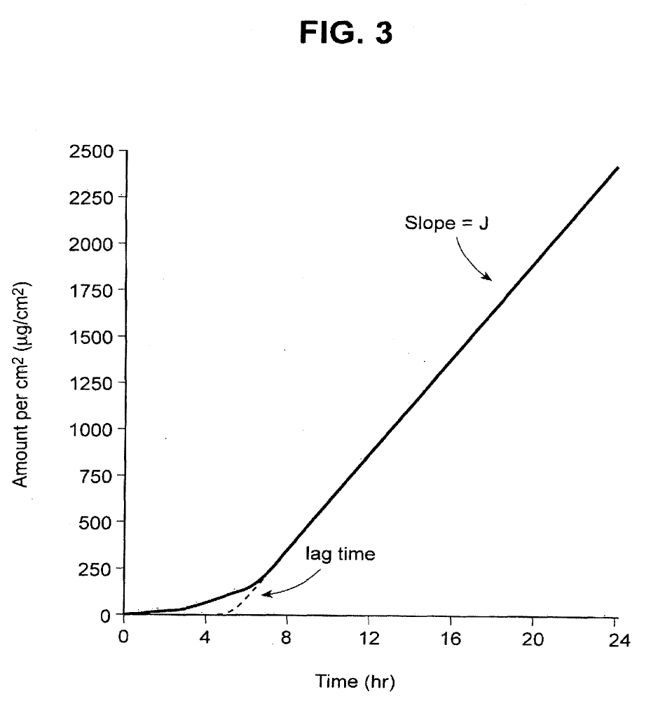

FIG. 4 depicts the cumulative amount of Compound (0.96% w/v) permeated across human cadaver skin from a vehicle system with or without CPE-215.

FIG. 5 depicts the cumulative amount of compound 0.96% w/w) permeated across human cadaver skin from a vehicle system containing IPM or CEP-215.

FIG. 6 depicts the cumulative amount of compound (0.96% w/w) permeated across human cadaver skin from vehicle containing varying levels of IPM.

### DETAILED DESCRIPTION OF THE INVENTION

### A) Compound

As noted above, all of the formulations of this invention contain the compound 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone whose structure is depicted below:

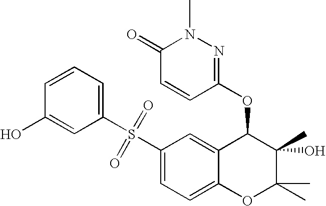

This compound is also commonly referred to as (3S,4R)-[6-(3-hydroxyphenyl)sulfonyl]-2,2,3-trimethyl-4-(2-methyl-3-oxo-2,3-dihydropyridazin-6-yl-oxy)-3-chromanol and (3S,4R)-3,4-dihydro-4-(2,3-dihydro-2-methyl-3-oxopyridazin-6-yl)oxy-3-hydroxy-6-(3-hydroxyphenyl)sulphonyl-2,2,3-trimethyl-2H-benzo[b]pyran). Example 7 of U.S. Pat. No. 5,912,244 discloses one method for producing the compound.

“Compound of the invention” and “Compound” are being used interchangeably and should be treated as synonyms. Each refers to 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone. Additionally, the term “Compound” shall at all times be understood to include all active forms of 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone, including, for example, the free form thereof, e.g., the free acid or base, and also all polymorphs, hydrates, solvates, tautomers, stereoisomers, e.g., diastereomers and enantiomers, and the like, and all pharmaceutically acceptable salts, and admixtures of such physical forms, unless specifically stated otherwise.

### B) Definitions

As used throughout this application, including the claims, the following terms have the meanings defined below, unless specifically indicated otherwise. The plural and singular should be treated as interchangeable, other than the indication of number.
a. “Reference Solution”—refers to a topical formulation containing a predefined concentration of the Compound (see section D for concentration), dissolved in a solution consisting of 70% ethanol/30% propylene glycol w/w.
b. “Mammal” includes humans, and primates such as stump-tailed macaques, companion animals such as dogs, cats, gerbils, etc. and livestock such as cattle, swine, horses, llamas, and sheep.
c. “Promoting hair growth” includes stimulating an increase in total hair mass and/or length. Such increase includes increased length and/or growth rate of hair shafts (i.e. follicles), increased number of hairs, and/or increased hair thickness (from vellus hair to full-thickness hair). Some or all of the above end results can be achieved by prolonging or activating anagen, the growth phase of the hair cycle, or by shortening or delaying the catagen and telogen phases. “Promoting hair growth” should also be considered to include preventing, arresting, decreasing, delaying and/or reversing hair loss.
d. “Alopecia,” as used herein, encompasses partial or full baldness, hair loss, and/or hair thinning.
e. “Treating or alleviating alopecia” refers to promoting hair growth in mammals that have experienced, or are considered at risk for experiencing, alopecia.
f. “Pharmaceutically acceptable” means suitable for use in or on mammals.
g. “Dermatologically acceptable” refers to substances, including the final formulations, which may be applied to the skin or hair.
h. “Pharmaceutically acceptable salts” is intended to refer to either “pharmaceutically acceptable acid addition salts” or “pharmaceutically acceptable basic addition salts”.
i. “Pharmaceutically acceptable acid addition salts” is intended to apply to any non-toxic organic or inorganic acid addition salt of the base compound represented by Formula I, or any of its intermediates. Illustrative inorganic acids, which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various hydrophilic organic solvents.
j. “Pharmaceutically acceptable basic addition salts” is intended to apply to any non-toxic organic or inorganic basic addition salts of the compound represented by Formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
k. The term “solvate” is a crystalline form of a compound or salt thereof, containing one or more molecules of a solvent of crystallization, i.e., the Compound or a salt thereof, containing solvent combined in the molecular form. An ethanol solvate of the Compound is a solvate in which the solvent is ethanol. A “hydrate” is a solvate in which the solvent is water.

### C) Flux

Drug absorption into the skin occurs by passive diffusion. The rate at which drug is transported across the stratum corneum follows Fick's Law of Diffusion. In other words, the rate of drug transport depends on the partition coefficient of drug between skin and formulation, diffusivity of drug through stratum corneum, drug concentration in the formulation, and the surface area of the skin to which it is exposed. It is inversely proportional to the thickness of the stratum corneum.

Experimentally, the flux can be determined by measuring the amount of drug permeation into the receptor compartment of a Franz cell over time. The cumulative drug permeated per cm² is than plotted against time and the steady state permeation rate (flux, J) is calculated from the slope of the linear portion of the curve according to Fick's 1^{st} law of diffusion.

In Examples 2-5 infra, the cumulative amount of Compound permeated through the skin as amount per surface area (μg/cm²) was plotted against time (hours). Flux (μg/cm²/hr) was determined by calculating the slope of the linear portion of the permeation profile. Drawing III depicts such a calculation. Typical variance in measured flux of 10 replicate samples is about 30-50%. However, if the formulation provides low flux of the Compound, indicating poor permeation through the skin, the variance could be much higher than the typical 30-50%.

### D) Franz Diffusion Methodology

The in-vitro human cadaver skin model has proven to be a valuable tool for the study of percutaneous absorption of topically applied compounds. This model is also commonly referred to as the Franz Diffusion Method. (Franz, T J, In Skin: Drug Application and Evaluation of Environmental Hazards, Current Problems in Dermatology, Vol. 7, G. Simon, Z. Paster, M. Klingberg, M. Kaye (Eds.), Basel, Switzerland, S. Karger, 1978, pp. 58-68.)

This methodology utilizes a device known as the Franz Diffusion Cell. A typical device is depicted in Drawing II. The test is carried out using the following general methodology. A sample of human cadaver skin is inserted in to the area labeled as skin. The skin should be oriented so that the stratum corneum is facing the Donor Compartment and the dermis is facing the Receptor Compartment. The Receptor Compartment will be filled with a predefined solution (based on solubility of test agent). A dose of test agent, in a defined volume, is inserted into the Donor Compartment so that it may come in contact with the skin sample and potentially diffuse thru the skin into the Receptor Compartment. Samples of solution are then withdrawn from Receptor Compartment at defined time points and the concentration of test agent is determined. The amount of test agent permeated per cm² of skin is plotted versus time and the flux of the test agent is calculated as described above. The reader's attention is directed to Example 2, which provides a more detailed discussion of this test.

As with any testing method, the results can vary depending upon how the test is carried out. Identical formulations containing identical concentrations of the same test agent can provide significantly different fluxes, if selected test parameters are not controlled. Thus, as used in this application including the claims, any flux determination should be carried by controlling the following parameters:
1) Apparatus—The apparatus should be a Franz Diffusion Cell. Each cell should have a cell volume of 5.4 to 5.5 ml. Ten (10) cells should be utilized per formulation (i.e. both the test formulation and the reference formulation). As is standard in the field, the results are reported as the mean.
2) Skin—Skin samples used for both the test formulation and the reference formulation should be human cadaver skin obtained from a single donor, who is male, between the ages of 50 and 75 and is obtained from the subjects back. The thickness of the skin should range from 700 microns to 1100 microns (average thickness should not vary by more than 20%). The surface area of the skin samples should be 0.635 cm² (internal diameter of opening of 9 mm). The donor compartment should be maintained at room temperature (see Table A for specifics).
3) Testing—Testing should be carried out for 24 hours. Sample should be withdrawn at the initiation of testing, and 2, 4, 12, 16, and 24 hours after initiation of testing. The sample volume should be 0.5 ml. The Compound should be present at identical concentrations in both the test formulation and the reference formulation (w/v for liquids and w/w for semi-solids). This concentration should be equal to that used in the final dosage form (i.e., what is being developed for eventual human application). A dose volume of 10 μl/0.635 cm² should be used.
4) Receptor Solution—0.1% (w/v) Brij-98 in Dulbecco's phosphate buffered saline, pit 6.9 (KH₂PO₄ 14.7 mM and Na₂HPO₄ 80.9 mM).
5) Barrier Integrity—The barrier integrity of human cadaver donor skin is assessed to ensure that changes in flux are attributed to the formulation versus a faulty skin barrier (e.g., cut, scrape, lesion, etc). Radiolabeled water, or mannitol, which does not readily permeate through human cadaver skin is applied on the epidermal side of the skin. The receptor fluid is assayed for these compounds. High levels of these compounds in the receptor fluid indicate a faulty barrier and the test skin should be discarded.
6) Assay method—Radioactivity

These variables are summarized below in Table A

TABLE A
Parameters For the Franz Diffusion Study*

Parameters

| | |
|---|---|
| Apparatus | Franz diffusion cells, Crown Glass Company |
| Number of Cells | 10 |
| Membrane | Human cadaver skin (50-75 year old male, back |
| | skin) Single Donor |
| Thickness | 700-1100 μm (should be within ±20%) |
| Duration | 24 Hours |
| Surface Area | 0.635 cm² (9 mm diameter) |
| Dose | 10 μL |
| Concentration | Equal for Test and Reference Formulations |
| Cell Volume | 5.4-5.5 mL (Depending on each individual cell) |
| Receptor Solution | 0.1% (w/v) Brij-98 in Dulbecco's phosphate |
| | buffered saline, pH 6.9 (KH₂PO₄ |
| | 14.7 mM and Na₂HPO₄ 80.9 mM) |
| Barrier Integrity | Same barrier function for each replicate |
| Donor Temperature | Room temperature and each cell should be within |
| | 0.5° C. of each other) |
| Dose volume | 10 μl/0.635 cm² |
| Receptor temperature | 37 ± 0.5° C. |

*Variables may differ from those in Examples 2-5 due to early stage of research project.

### E) Formulations

As noted above, the invention is directed to a topical formulation which comprises (a) the Compound, (b) a dermatologically acceptable carrier, and (c) exhibits a flux of said Compound across human cadaver skin into a receptor compartment of a Franz Diffusion cell that is at least three times higher than the flux exhibited by a reference formulation (i.e. same concentration of Compound in 70% ethanol/30% propylene glycol w/w). In another embodiment, said formulation exhibits a flux of said compound across human cadaver skin into a receptor compartment of a Franz Diffusion cell that is at least five times higher than the flux exhibited by the reference formulation. In a further embodiment, said formulation exhibits a flux of said compound across human cadaver skin into a receptor compartment of a Franz Diffusion cell that is at least ten times higher than the flux exhibited by the reference formulation.

The formulation type, is not critical to the invention. It may be in the form of aqueous, alcoholic or aqueous-alcoholic solutions, or in the form of creams, gels, emulsions or mousses, or alternatively in the form of aerosol compositions also comprising a propellant under pressure. The composition according to the invention can also be a hair care composition, and in particular a shampoo, a hair-setting lotion, a treating lotion, a styling cream or gel, a dye composition, a lotion or gel for preventing hair loss, etc.

The quantity of Compound contained in the formulation is not critical. A sufficient quantity should be utilized to promote hair growth in a mammal, especially a human. This quantity can range from 0.001 to about 10% (w/w) for semi-solid dosage forms. In a further embodiment, this quantity is from about 0.01% to about 5% (w/w). In yet another embodiment, this quantity is from about 0.5% to about 3% (w/w). In a typical embodiment, this quantity is from about 0.5% to about 1.5% (w/w). For liquid dosage forms, the quantities above should be expressed as % w/v.

In addition to the Compound, the formulation will contain at least one carrier. As used herein, a carrier refers to one, or more, semi-solid, or liquid fillers, diluents, vehicles, etc. that are suitable for topical administration on a human. The amount of carrier employed in the formulations may vary and will depend, for example, on the particular carrier(s) employed, the quantity of the Compound employed, and the like. The carrier may be present in the formulations in an amount ranging from about 1% (w/w) to about 99.9% (w/w), and all combinations and sub-combinations of ranges and specific amounts therein. In one embodiment, the carrier may be employed in the present formulations in an amount of from about 20% (w/w) to about 99% (w/w), with concentrations ranging from about 30% (w/w) to about 90% (w/w) being particularly useful.

In addition to a carrier, the formulation may optionally contain a penetration enhancer. A penetration enhancer is a substance, which promotes the dermal absorption of a test agent. Such compounds are also often referred to as accelerants or absorption promoters. The quantity of penetration enhancer may vary and is not critical to the invention. It may be present in the quantity of about 0.1% to about 40% (w/w). The penetration enhancer may also be present in the quantity of about 0.5% to about 30% (w/w), or alternatively about 5% to about 25% (w/w). In yet another embodiment, the formulation comprises 20% (w/w) of the penetration enhancer.

Examples of such penetration enhancers include hydrocarbons (e.g. n-nonane, n-decane, squalane), alkanols and alkenols (e.g. ethanol, propanol, butanol, polyethylene glycols, propylene glycol, lauryl alcohol, transcutol, glycerine, oleyl alcohol), acids (e.g. oleic acid, linoleic acid, lauric acid, myristic acid, palmitic acid, stearic acid, α-hydroxyl acids, β-hydroxyl acids), esters (e.g. isopropyl myristate, propylene glycol, dicaprylate/dicaprate, dibutyl adipate, methyl salicylate, glyceryl monooleate, glyceryl monocaprylate, glyceryl caprylate/caprate), alkyl amino esters (e.g. decyl-(N,N-dimethylamino)isopropionate, myristyl-(N,N-dimethylamino)isopropionate, dodecyl-(N,N-dimethylamino)propionate, dodecyl-(N,N-dimethylamino)acetate), amides (e.g. N,N-diethyl-m-toluamide), urea, amino acids (e.g. valine), aromatic compounds (e.g. thymol), sulfoxides (e.g. decylmethyl sulfoxide), terpenes (e.g. α-terpinene, d-limonene, menthol), pyrrolidone and imidazole derivatives (e.g. N-methyl-pyrrolidone), 1-dodecyl-hexahydro-2H-azepin-2-one, cyclopentadecanolide, salicylates and many others as listed in Transdermal and Topical Drug Delivery Systems (Eds. Ghosh, T. K., and Pfister, W. R.) and Drug Permeation Enhancement (Eds. Hsieh, D. S.).

In one embodiment, the formulation is an alcoholic solution. In such a formulation, the carrier is typically an admixture of monohydroxy alcohols and polyols. The formulation may optionally contain at least one penetration enhancer.

Examples of suitable monohydroxy alcohols include, for example, ethanol, propanol, butanol and benzyl alcohol. Reference herein to “ethanol” includes absolute alcohol, as well as “alcohol USP” and all denatured forms of 95% ethanol. As used herein, the term “propanol” refers to all isomeric forms, including n-propanol and isopropanol, and the term “butanol” refers to all isomeric forms, including, for example, n-butanol, iso-butanol and sec-butanol. In one embodiment, the alcohol is selected from the group comprising ethanol, isopropyl alcohol, and benzyl alcohol, with ethanol being particularly useful.

Examples of suitable polyols include, for example, propylene glycol, dipropylene glycol, hexylene glycol, 1,3-butylene glycol, liquid polyethylene glycols, such as polyethylene glycol 200 (PEG-200) and polyethylene glycol 400 (PEG-400). A particularly useful polyol is propylene glycol.

For those formulations that are alcoholic solutions or aqueous-alcoholic solutions, the polyol will typically be present in the quantity of from about 0 to about 80% w/w, more typically about 10 to about 25% w/w. The monohydroxy alcohol will be present in the quantity of about 10 to about 99.9% w/w, more typically from about 40 to about 90% w/w. One example of such an alcoholic solution is a formulation containing about 1% w/v of Compound, about 10 to 30% w/w of a polyol, and about 40 to about 90% w/w of a monohydroxy alcohol. Minor amounts of water can also be included in the formulation.

Optionally a penetration enhancer may be incorporated into these alcoholic solutions. In one embodiment, the formulation contains from about 10% to about 25% (w/w) of a polyol, from about 50% to about 70% (w/w) of a monohydroxy alcohol and from about 1% to about 30% (w/w) of a penetration enhancer. In a second embodiment, the formulation contains from about 10% to about 25% (w/w) of a polyol selected from the group consisting of propylene glycol, dipropylene glycol, hexylene glycol, 1,3-butylene glycol, polyethylene glycol, and glycerol, from about 50% to about 70% (w/w) of a monohydroxy alcohol selected from the group consisting of ethanol, isopropyl alcohol and benzyl alcohol and from about 1% to about 30% (w/w) of a penetration enhancer selected from the group consisting of isopropyl myristate, cyclopentadecanolide and propylene glycol dicaprylate/dicaprate. In a more specific embodiment, the formulation contains from about 10% to about 25% (w/w) of propylene glycol, from about 50% to about 70% (w/w) of ethanol, and from about 1% to about 30% (w/w) of isopropyl myristate. More specifically, the formulation contains about 0.5 to about 3 w/v % of Compound, about 20% (w/w) of a propylene glycol, about 60% (w/w) of ethanol and about 20% (w/w) of isopropyl myristate.

In addition to solutions, the formulations may also be semi-solids such as creams, ointments, or gels. The quantity of Compound contained within these semi-solids will vary, but will typically range from about 0.5% w/w to about 3% w/w, more typically about 1% w/w.

Gels are formed by the entrapment of large amounts of aqueous or aqueous-alcoholic liquids in a network of colloidal solid particles (collectively the carrier). These colloids are typically present at concentrations of less than 10% w/w and are also referred to as gelling agents. Examples of suitable gelling agents include carboxymethyl cellulose, hydroxypropylmethyl cellulose, hydroxyethyl cellulose, methyl cellulose, sodium alginate, alginic acid, pectin, tragacanth, carrageen, agar, clays, aluminium silicate, carbomers, etc. The aqueous-alcoholic solution can be similar to those described above, except that water content may be up to 60% w/w, with a corresponding decrease in alcohol content.

Creams may also be utilized. They are emulsions of oleaginous substances and water (i.e. the carrier). There are two types of creams. The first is a water-in-oil cream “w/o” in which an aqueous phase is dispersed in an oil phase. These can be prepared by mixing equal parts of wool alcohol ointment and water. Alternatively, they can be prepared from beeswax, an admixture of beeswax and mineral oil, or an admixture of beeswax and vegetable oil. Additional details regarding such formulations may be found in Drugs and the Pharmaceutical Sciences, Volume 18, Dermatological Formulations Percutaneous Absorption, Brian Barry, (1983) at page 314.

Oil-in-water creams (o/w) have an oil dispersed within an aqueous base. O/W creams are typically invisible after applications, wash off and are popular with consumers. The oil phase of such creams typically contain up to about 20% w/w of stearic acid, long chain waxy alcohols, vegetable oils, or waxes. An aqueous phase, containing the Compound, emollients, stabilizers, anti-oxidants, etc., constitutes the remaining components. The Compound may also be incorporated directly into commercially prepared cream bases such as Aqueous Cream BP, Cetrimide Cream BP, Cetomacrogol BP or Dimethicone BP as well. Further details on o/w creams may be found at Barry, supra at pages 314-322.

Ointments are another semi-solid dosage form that may be utilized. Traditional ointment bases (i.e. the carrier) include hydrocarbons (petrolatum, beeswax, etc.) vegetable oils, fatty alcohols (cholesterol, lanoilin, wool alcohol, stearyl alcohol, etc.) or silicones. Further details on ointments may be found at Barry, supra at pages 304-312.

Pastes are basically ointments, into which a high percentage of insoluble particulate solids have been added, up to 50% by weight. Insoluble solids such as starch, zinc oxide, calcium carbonate, or talc may be used. The ointments can be prepared as above. Further details on pastes may be found at Barry, supra at page 322.

Aerosols may also be utilized. The Compound may be dissolved in a propellant and a co-solvent such ethanol, acetone, hexadecyl alcohol, etc. Foaming agents may be incorporated to produce a mousse. Further details on aerosols may be found at Barry, supra at page 323-324.

A wide variety of methods may be used for preparing the formulations described above. Broadly speaking, the formulations may be prepared by combining together the components of the formulations, as described herein, at a temperature and for a time sufficient to provide a pharmaceutically effective and elegant composition. The term “combining together”, as used herein, means that all of the components of the compositions may be combined and mixed together at about the same time. The term “combining together” also means that the various components may be combined in one or more sequences to provide the desired product. The formulation can be prepared on a weight/weight (w/w) or a weight/volume (w/v) basis depending upon the form of the final dosage form.

The formulations may be packaged for retail distribution directly to the consumer (i.e., an article of manufacture or kit). Such articles will be labeled and packaged in a manner advising the patient how to use the product to promote hair growth. Such instructions will include the duration of treatment, dosing schedule, precautions, etc. These instructions may be in the form of pictures, written instructions, or a combination thereof. They may be printed on the side of the packaging, be an insert, or any other form of communication appropriate for the retail market.

### F) Therapeutic Use

The formulations of the present invention may be used to promote hair growth. It will typically be applied from 1 to 4 times daily or less frequently, as recommended by a physician. In one embodiment, the formulations may be used to treat or prevent alopecia. The most common type of alopecia is androgenetic alopecia. This condition is also commonly referred to as male pattern baldness and female pattern baldness. Other types of alopecia may also be treated by the formulation of the present invention.

Anagen effluvium is hair loss due to chemicals or radiation, such as chemotherapy or radiation treatment for cancer. It is also commonly referred to as “drug induced” or “radiation induced” alopecia. The formulations may be used to treat this condition.

Alopecia areata is an autoimmune disorder, which initially presents with hair loss in a rounded patch on the scalp. It can progress to the loss of all scalp hair, which is known as alopecia totalis and to the loss of all scalp and body hair, which is known as alopecia universalis. The formulations may be utilized to treat or prevent these types of alopecia.

Traumatic alopecia is the result of injury to the hair follicle. It is also commonly referred to as “scarring alopecia”. Psychogenic alopecia occurs due to acute emotional stress. By inducing anagen, the compound can be beneficial in these types of alopecia as well. Thus, the invention should not be construed as being limited to treating androgenetic alopecia. The formulations may be used to alleviate any type of hair loss.

In a further embodiment, the formulation comprising the Compound can also be used in patients who have not yet experienced hair loss, but believe that they are at risk of experiencing alopecia. Examples of such patients include those who will be undergoing cancer chemotherapy with a drug regimen known to induce alopecia. Young adults experiencing mental distress at the thought of balding, especially those with a family history of baldness, may also benefit from such prophylactic treatment. Such prophylactic treatment is encompassed by the term “promoting hair growth”.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention. The following examples and biological data are being presented in order to further illustrate the invention. This disclosure should not be construed as limiting the invention in any manner.

### EXAMPLES

### Example 1

### Preparation of Topical Formulations Comprising the Compound with Ethanol, Propylene Glycol and Isopropyl Myristate using the w/v and the w/w Methods

### Example 1A

### W/V Method

A formulation within the scope of the present invention and containing 0.96% w/v of the Compound was prepared as follows:

The components set forth in Table I was added to a conical tube in the sequence listed in table and vortexed until the mixture was homogeneous. Approximately 65.9 mg of the solvate form of the Compound was weighed in a glass vial. Six milliliters of the carrier solution shown in Table I was added to the vial to give approximately 0.96% w/v of the Compound in the formulation.

TABLE I

| Component | Amount (g) | Amount (% w/w) |
|---|---|---|
| Propylene glycol | 2 | 20 |
| Isopropyl myristate | 2 | 20 |
| Ethanol 200 Proof | q.s. to 10 | q.s. to 100 |

Carrier solutions for the formulations provided on Tables II through IV were prepared in accordance with the procedure set forth above.

### Example 1B

### W/V Method

A formulation within the scope of the present invention and containing approximately 1% of the Compound was prepared as follows:

Approximately 11 mg of the solvate form of the Compound was weighed in a glass vial. One milliliter of the carrier solution shown on Table II was added to the glass vial to give approximately 0.96% w/v of the Compound.

TABLE II

| Component | Amount (g) | Amount (% w/w) |
|---|---|---|
| Propylene glycol | 2 | 20 |
| Isopropyl myristate | 2 | 20 |
| Water | 0.5 | 5 |
| Ethanol 200 Proof | q.s. to 10 | q.s. to 100 |

### Example 1C

### W/V Method

A formulation within the scope of the present invention and containing 0.96% of the Compound was prepared as follows:

The Compound (11 mg) was weighed in a glass vial. One mL of the carrier solution as shown on either Table III or IV and prepared as above was added to the glass vial to give a formulation comprising approximately 0.96% w/v of the Compound.

TABLE III

| Component | Amount (g) | Amount (% w/w) |
|---|---|---|
| Propylene glycol | 2 | 20 |
| Isopropyl myristate | 1.5 | 15 |
| Benzophenone-3 | 0.3 | 3 |
| Ethanol 200 Proof | q.s. to 10 | q.s. to 100 |

TABLE IV

| | Formulation 4 | Formulation 5 |
|---|---|---|
| Components | (% w/w) | (% w/w) |
| Propylene glycol | 20 | 10 |
| Isopropyl myristate | 10 | 20 |
| Ethanol 200 Proof | q.s. to 100 | q.s. to 100 |

### Example 2

### Method for Conducting In-Vitro Human Cadaver Skin Permeation Studies

In-vitro Percutaneous absorption was measured using a Franz diffusion cell system. The cell volume of Franz diffusion cell was 5.5 to 5.7 mL, the donor surface area was 0.635 cm², and the internal diameter of opening was 9 mm. Cryopreserved, human cadaver skin having the subcutaneous layer removed was obtained from Ohio Valley Tissue and Skin Center and stored at −65° C. until use. Prior to the experiment, skin was thawed at room temperature. Receptor compartment was filled completely with 0.1% w/v Brij-98 in Dulbecco's phosphate buffered saline, pH 6.9 (KH₂PO₄ 14.7 mM and Na₂HPO₄ 80.9 mM). The receptor compartment was maintained at 37° C. using a water bath and was stirred using a magnetic stirrer. Donor compartment was left at room temperature. A ⅞″ circular piece of human cadaver tissue was removed using a hollow punch and placed on the cell with the epidermal side facing the donor compartment and the dermal side facing the receptor compartment. The donor cap was then clamped onto the cell. Any bubbles introduced at the dermis/receptor interface were removed by inverting the cell.

In tests where radioactive Compound was used, fifteen microliters of ³H Compound having a specific activity of 15 Ci/mmol dissolved in ethanol was transferred in an eppendorf tube. Compound was dried by passing N₂ gas over the solution. One hundred fifty microliters of a formulation prepared in Example 1 comprising 0.96% w/v of the Compound was added to the radioactive Compound. The contents were mixed using a vortex.

The skin was equilibrated for a period of 30 minutes before applying the dose. 10 μL (over a dosing area of 0.635 cm²) of approximately 0.96% w/v Compound solution was applied onto the skin using a positive displacement pipette. In case of radioactive Compound specific activity in 10 μL was approximately 25-30 microCi/mg. A 0.5 mL of the sample was removed from the sample port of the receptor compartment at approximately 0, 2, 5, 8, 18, and 24 hours and replaced by an equal volume of the receptor solution. Any bubbles introduced at the receptor/dermis interface were removed by inverting the cells.

Following the last sample, the surface of the skin was washed four times with 0.5 mL (each time) of ethanol. Following alcohol washes, the dosed surface was dabbed with a dry cotton swab, the donor cap removed and the skin surface was swabbed twice with cotton swab wetted with ethanol and finally the skin surface was dabbed with the dry cotton swab. All the swabs and ethanol washes were placed in a conical tube. The donor cap was placed in the conical tube.

The skin was removed from the cells, and epidermal part of the dosed area was separated by scrapping and placed in a tared scintillation vial. A punch biopsy was taken through the dermis and placed in a tared scintillation vial. Weights of dermis and epidermis were recorded. The excess skin portion was either placed in scintillation vial or added to the conical tube containing surface wash.

### Preparation and Analysis of the Samples:

### Treatment of Samples for Analysis by Radioactivity:

Receptor samples: 15 mL of scintillation liquid were added to the receptor samples and radioactivity of the samples measured.
Surface wash: 23 mL of ethanol were added to the conical tube containing alcohol washes, swabs, and donor cap. All the tubes were vortexed for 45 seconds and sonicated for 15 minutes. 0.5 mL of the above solution was transferred into a scintillation vial; 15 mL of scintillation liquid were added to the vial and radioactivity measured.
Epidermis samples: 1 mL of Solvable™ was added to the vial containing epidermis, the samples were kept at 60° C. in a water bath set at 30 RPM until the tissue dissolves (less than 20 hours). The vials were allowed to cool down and 15 mL of scintillation liquid (Ultima Gold XR) were added to the vial. The samples were let to stand for 3-4 hours in dark before measuring the radioactivity.
Dermis samples: 2 mL of Solvable™ were added to the vial containing dermis, the samples were kept at 60° C. in a water bath set at 30 RPM until the tissue dissolves (less than 24 hours). The vials were allowed to cool down and 15 mL of scintillation liquid were added to the vial. The samples were let to stand for 3-4 hours in dark before measuring the radioactivity.
Excess skin: The excess portion of the skin was divided into two pieces and each placed in a separate scintillation vial. To each vial, 3 mL of Solvable™ were added. The samples were kept at 60° C. in a water bath set at 30 RPM until the tissue dissolves (approximately 24 hours). The vials were allowed to cool down and 15 mL of scintillation liquid were added to the vial. The samples were let to stand for 3-4 hours in dark before measuring the radioactivity.

Samples were analyzed using Liquid Scintillation Analyzer (TriCarb 2500 TR from Perkin Elmer).

### Calculation of Flux and Dermal Amounts of the Compound:

Flux calculation: Amount of the Compound permeated through the skin as amount per surface area (μg/cm²) was plotted against time (hours). Flux (μg/cm²/hr) was determined by calculating the slope of the linear portion of the permeation profile. Calculation of amounts of the Compound in various skin strata and receptor compartment:

Quantitation of Compound Penetration by Radioactive Analysis

Samples collected in were counted in a scintillation counter and the percent of radioactive Compound detected in each skin layer and the receptor compartment represented the amount of Compound that was in the specific compartment at the time the sample was removed. For instance, to determine the penetration of the Compound at a fixed time period (24 hours), the amount of radioactivity in each layer was determined at 24 hrs following application of the ³H-Compound. The amount of Compound on the skin surface represented the amount of Compound that did not penetrate the stratum corneum over the 24 hr period. The amount of radioactive Compound recovered in the epidermal sample represented the amount of Compound that penetrated the stratum corneum by that time point, but did not penetrate to the dermis. The amount of Compound in the dermis, represented the amount of Compound that penetrated through the stratum corneum and the epidermis during the 24 hr period and was retained in the dermis layer. The amount of Compound in the receptor compartment represented the amount of Formula Compound that had penetrated through the stratum corneum, the epidermis and the dermis during the 24 hr test period.

Samples were treated and analyzed as described above. From the amount of Compound mean percent of the Compound in samples were calculated based on total amount of the Compound applied topically.

### Example 3

### Comparison of Flux Rates of the Compound in a Carrier Comprising 63.5% ethanol: 20% propylene glycol: 8% Cyclopentadecanolide (CPE-215): 7.5% Water (w/w/w/w) Compared to a Carrier Containing 70% ethanol: 30% propylene glycol (w/w)

In order to measure the effectiveness of penetration enhancers to increase the rate of penetration of the Compound through human skin, the flux rates of the Compound in two different carriers was determined using the Franz Cell Diffusion system described in Example 2. The thickness of the human skin was 0.71-0.96 mm and 0.90-1.04 mm. The penetration of the Compound in the reference formulation comprising 0.96% w/v of the Compound or in a carrier comprising ethanol:propylene glycol:Cyclopentadecanolide:water (63.5:20:8:7.5% w/w/w/w) was compared.

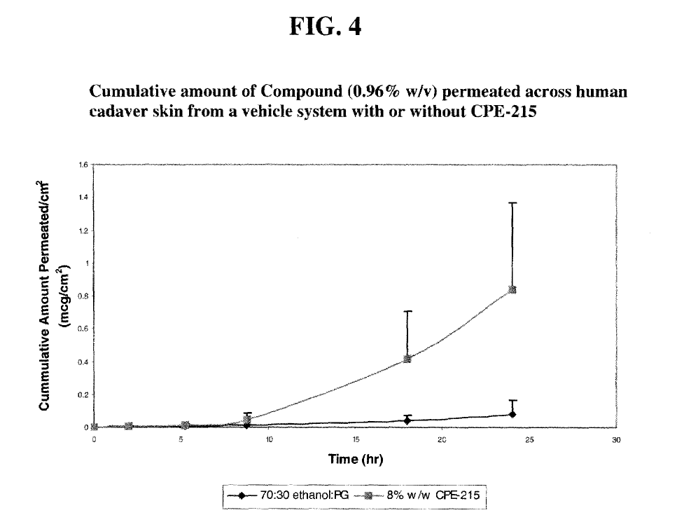

The results represented in FIG. 4 indicate that the skin permeation of Compound was greatly enhanced in a carrier comprising a penetration enhancer, as compared with the reference formulation. FIG. 4 demonstrates that Compound appears in the receptor medium after a certain time, defined as lag time. The lag time is a result of the time required for the Compound to cross the primary barrier in the skin, the stratum corneum, and then diffuse through the epidermis and dermis before entering the receptor phase. The flux of the Compound applied in the reference formulation was 0.004±0.006 μg/cm²/hr. The flux of the Compound applied in a carrier also comprising a penetration enhancer (ethanol:propylene glycol:Cyclopentadecanolide:Water (63.5:20:8:7.5% w/w/w/w) was 0.053±0.003 μg/cm²/hr. Thus, the formulation comprising the penetration enhancer Cyclopentadecanolide provided a higher flux of the Compound through layers of the skin than did the reference formulation.

TABLE 2
Effect of Cyclopentadecanolide on the In Vitro Human
Cadaver Skin Permeation of Compound (0.96% w/v)

| | Mean Percentage of Radioactivity Recovered at | | | | |
|---|---|---|---|---|---|
| | 24 Hours (±SD) | | | | |
| Vehicle | Epidermis | Dermis | Receptor | Surface | Total |
| 70:30% w/w | 2.53 | 0.06 | 0.05 | 79.30 | 81.94 |
| EtOH/PG | (1.04) | (0.03) | (0.06) | (5.65) | (4.68) |
| 63.5:20:8:7.5% | 8.50 | 0.61 | 0.56 | 84.77 | 94.43 |
| w/w | | | | | |
| EtOH/PG/CPE-215/ | (1.92) | (0.36) | (0.35) | (3.98) | (2.80) |
| Water | | | | | |

SD = Standard deviation.

### Example 4

### The Flux Obtained when IPM was used as the Penetration Enhancer was Comparable to that Obtained with Cyclopentadecanolide as the Penetration Enhancer in the Franz Cell Diffusion Assay Described in Example 2

Carriers comprising the Compound and either ethanol:propylene glycol:IPM (60:20:20% w/w/w) or ethanol:propylene glycol:Cyclopentadecanolide:Water (63.5:20:8:7.5% w/w/w/w) were prepared by the method described in Example 1. The flux rate was determined as described in Example 2. The skin thickness in this study was 0.71-0.96 mm and 0.90-1.04 mm.

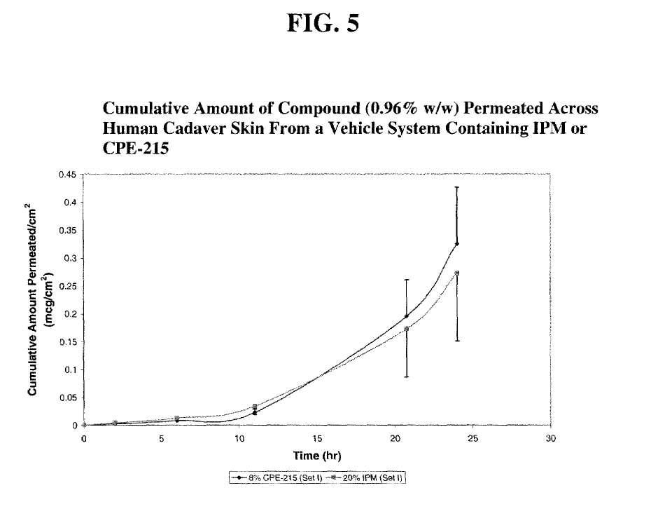

FIG. 5 represents cumulative amounts of Compound absorbed through the skin into the receptor at various time points. The flux of the Compound applied in a carrier comprising ethanol:propylene glycol:IPM (60:20:20% w/w/w) was 0.017±0.008 μg/cm²/hr. The flux rate of the Compound applied in a carrier comprising cyclopentadecanolide (ethanol:propylene glycol:Cyclopentadecanolide:Water (63.5:20:8:7.5% w/w/w/w) was 0.022±0.007 μg/cm²/hr. The formulation comprising IPM provided a similar flux of the Compound through the skin as did the formulation comprising cyclopentadecanolide penetration enhancer.

The penetration of Compound measured in separate layers of the skin and in the receptor compartment after a fixed period of time (24 hrs) is represented in Table 3.

TABLE 3
Effect of the Vehicle System Containing IPM
or CPE-215 on the In Vitro Human Cadaver Skin
Permeation of Compound (0.96% w/v)

| | Mean Percentage of Radioactivity Recovered at | | | | |
|---|---|---|---|---|---|
| | 24 Hours (±SD) | | | | |
| Vehicle | Epidermis | Dermis | Receptor | Surface | Total |
| 60:20:20% w/w | 8.04 | 0.78 | 0.14 | 79.28 | 88.24 |
| EtOH/PG/IPM | (1.80) | (0.18) | (0.07) | (3.24) | (4.40) |
| 63.5:20:8:7.5% | 8.71 | 0.76 | 0.16 | 79.36 | 88.99 |
| w/w | | | | | |
| EtOH/PG/CPE-215/ | (2.88) | (0.16) | (0.07) | (6.06) | (6.24) |
| Water | | | | | |

SD = Standard deviation.

### Example 5

### Effect of Varying the Amount of IPM on the Skin Permeation of Compound

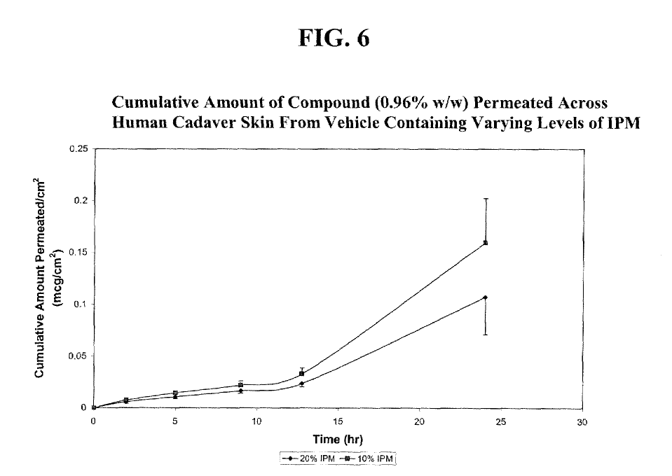

The effect of varying the level of IPM in the carrier on the penetration of the Compound was tested according to the method described in Example 2 using 1.03-1.19 mm thick human cadaver skin. FIG. 6 depicts the cumulative amount of Formula Compound absorbed through the skin into the receptor at various time points. The flux of the Compound applied in a carrier consisting of ethanol:propylene glycol:IPM (60:20:20% w/w/w) was 0.011±0.003 μg/cm²/hr. The flux of the Compound applied in a carrier comprising ethanol:propylene glycol:IPM (70:20:10% w/w/w) was 0.007±0.003 μg/cm²/hr.

TABLE 4
Effect of IPM Level on the In Vitro Human Cadaver
Skin Permeation of Compound (0.96% w/v)

| | Mean Percentage of Radioactivity Recovered at | | | | |
|---|---|---|---|---|---|
| | 24 Hours (±SD) | | | | |
| Vehicle | Epidermis | Dermis | Receptor | Surface | Total |
| 60:20:20% w/w | 12.00 | 1.00 | 0.07 | 73.00 | 86.1 |
| EtOH/PG/IPM | (2.30) | (0.50) | (0.02) | (2.10) | (1.60) |
| 70:20:10% w/w | 12.27 | 1.04 | 0.10 | 75.24 | 88.66 |
| EtOH/PG/IPM | (0.55) | (0.36) | (0.03) | (1.81) | (1.48) |

SD = Standard deviation.

### Example 6

### Comparison of Various Penetration Enhancers on the Penetration of Compound

Several other formulations according to the present invention comprising penetration enhancers were tested to determine the effect on the penetration of the Compound. The penetration of Compound (1.86% w/v) in the following carriers in the assay described in Example 2 was examined. When the Compound was administered in a carrier comprising EtOH/PG/DMI (dimethyl isosorbide) (60:30:10% w/w), the flux was 0.036±0.0336 μg/cm²/hr. The flux of Compound applied in the carrier ethanol:propylene glycol:IPM (50:30:20% w/w/w) was 191±0.026 μg/cm²/hr. The flux of Compound applied in a carrier consisting of (EtOH/PG/Miglyol 840 (50:30:20% w/w/w) was 0.11±0.007 μg/cm²/hr.

While direct comparisons were not carried out in each instance, the trend shows that formulations comprising penetration enhancers increased the flux of the Compound across human cadaver skin into the receptor when compared to the reference formulation.

What is claimed is:

1. A topical formulation comprising (a) an effective amount of 6-[[(3S,4R)-3,4-dihydro-3-hydroxy-6-[(3-hydroxyphenyl)sulfonyl]-2,2,3-trimethyl-2H-1-benzopyran-4-yl]oxy]-2-methyl-3(2H)-pyridazinone, or a pharmaceutically acceptable salt thereof, (b) a dermatologically acceptable carrier, and c) said formulation exhibits a flux of said compound across human cadaver skin in a Franz Diffusion cell test that is at least equal to or greater than three times the flux provided by a reference formulation.

2. The topical formulation of claim 1 in which said formulation exhibits a flux of said compound across human cadaver skin in a Franz Diffusion cell test that is at least equal to or greater than five times the flux provided by the reference formulation.

3. The topical formulation of claim 1 in which said formulation exhibits a flux of said compound across human cadaver skin in a Franz Diffusion cell test that is at least equal to or greater than ten times the flux provided by the reference formulation.

4. The topical formulation according to any one of claims 1, 2, or 3 in which said formulation is a liquid.

5. The topical formulation according to anyone of claims 1, 2, 3 or 4 in which said compound is present in the quantity of about 0.5% to about 3% (w/v).

6. The topical formulation according to anyone of claims 1, 2, 3, or 4 in which said formulation is a alcoholic or aqueous-alcoholic solution.

7. The topical formulation according to anyone of claims 1, 2, 3, 4, 5 or 6 in which said formulations contains from about 10% to about 25% (w/w) of a polyol, from about 50% to about 70% (w/w) of a monohydroxy alcohol and from about 1% to about 30% (w/w) of a penetration enhancer.

8. The topical formulation according to anyone of claims 1, 2, 3, 4, 5, 6 or 7, in which said formulation contains from about 10% to about 25% (w/w) of a polyol selected from the group consisting of propylene glycol, dipropylene glycol, hexylene glycol, 1,3-butylene glycol, polyethylene glycol, and glycerol, from about 50% to about 70% (w/w) of a monohydroxy alcohol selected from the group consisting of ethanol, isopropyl alcohol and benzyl alcohol and from about 1% to about 30% (w/w) of a penetration enhancer selected from the group consisting of isopropyl myristate, cyclopentadecanolide, propylene glycol dicaprylate/dicaprate.

9. The formulation according to anyone of claims 1, 2, 3, 4, 5 or 6 wherein said formulation comprises
(a). about 60% ethanol (w/w),
(b) about 20% propylene glycol (w/w), and
(c). about 20% (w/w) isopropyl myristate.

10. The formulation according to anyone of claims 1-3 in which said dosage form is a semi-solid dosage form selected from the group consisting of cream, gel, ointment, and paste.

11. The formulation according to claim 10 in which said compound is present in the quantity of about 0.5 to about 3% w/w.

12. The formulation according to claim 10 in which said formulation contains a penetration enhancer in the amount of about 1 to 30% w/w.

13. Use of the formulation of any of claims 1-12 to promote hair growth in a mammal.

14. An article of manufacture comprising a formulation according to anyone of claims 1-12 in which the formulations is packaged for retail distribution advising the patient how to use the product to promote hair growth.
